(19) United States
(12) Plant Patent Application Publication
(10) Pub. No.: US 2002/0088033 P1
(43) Pub. Date: Jul. 4, 2002

(54) ALSTROEMERIA PLANT NAMED 'STALOG'

(76) Inventor: C.A. HOOGENDOORN, NIEUWKOOP (NL)

Correspondence Address: CHRISTIE PARKER & HALE LLP, PO BOX 7068, PASADENA, CA, 911097068

(21) Appl. No.: 09/349,611

(22) Filed: Jul. 8, 1999

(30) Foreign Application Priority Data
Oct. 28, 1997 (NL) INC 595

(51) Int. Cl. A01H 5/00

(52) U.S. Cl. PLT/309

(57) ABSTRACT

A new and distinct Alstroemeria plant variety producing white flowers of good quality and attractive coloration.

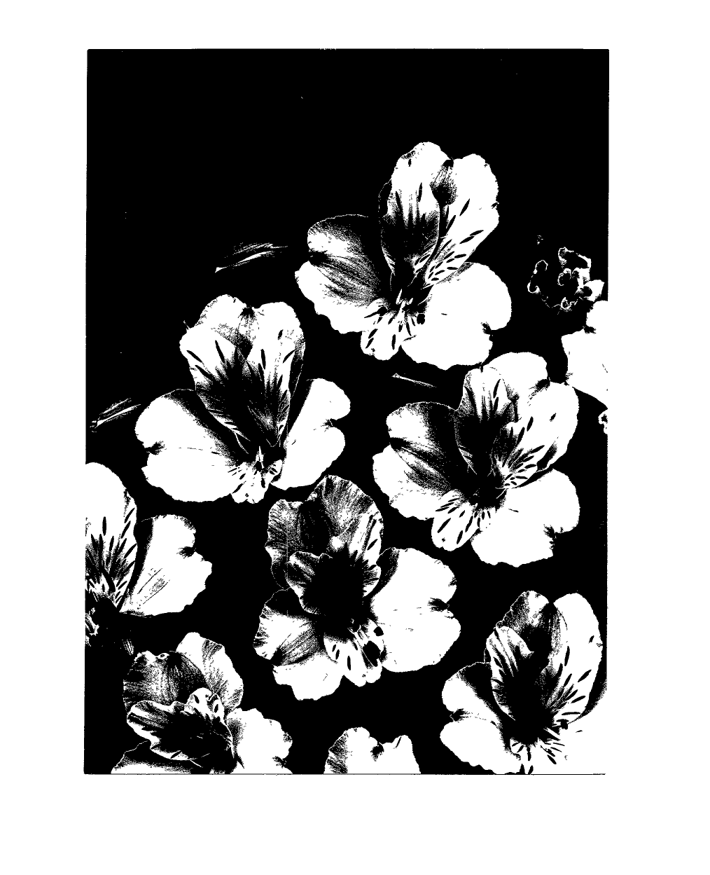

### BACKGROUND OF THE INVENTION

The present invention relates to a new and distinct variety of Alstroemeria which is a cross of two unnamed, undistributed seedlings; namely, 89W875-1 as seed parent and 87G1069-2 as pollen parent. The varietal denomination of the new variety is ‘Stalog’.

Asexual reproduction by dividing rhizomes, and by tissue culture, through successive generations in Aalsmeer, The Netherlands, has demonstrated that the combination of characteristics as herein disclosed for ‘Stalog’ are firmly fixed and retained through successive generations.

### SUMMARY OF THE INVENTION

The new variety of Alstroemeria, ‘Stalog’, produces white flowers of good quality.

### DESCRIPTION OF ILLUSTRATION

The new variety of Alstroemeria hybrid is illustrated in the accompanying illustration, which shows typical flower characteristics, with colors being as nearly true as is possible for illustrations of this type.

### DETAILED DESCRIPTION OF THE NEW VARIETY

The following is a detailed description of the new cultivar of Alstroemeria hybrid as observed in a greenhouse in Aalsmeer, The Netherlands. Color designations indicated are in accordance with The Royal Horticultural Society Color Chart by color plate designations.

‘Stalog’ has not been observed under all possible environmental conditions. Phenotypic expression may vary with variations in environment such as temperature, light intensity, day length and growing and cultural conditions.

| | |
|---|---|
| | Family: Amaryllidaceae |
| Botanical Classification: | Genus: Alstroemeria sp. |

PLANT

| | |
|---|---|
| Form: | Bush |
| Growth: | Vigorous |
| Height: | About 160-200 cm |
| Main stem: | About 35 cm |
| Branching | Flower stems do not branch |
| Characteristics: | |
| Flower Stem: | |
| Length: | About 160-200 cm |
| Thickness: | About 3-8 mm |
| Color: | Near 137A |
| Foliage | |
| Quantity: | Abundant |
| Number of leaves: | About 25-30 |
| Size of leaf | |
| Length: | About 10-15 cm |
| Width: | About 2-4 cm |
| Shape of leaf: | Elliptic |
| Margin: | Minutely crimped |
| Texture: | Glossy |
| Color | |
| Upper side: | Near 137A |
| Underside: | Near 137C |
| Stem color: | Near 137A |
| Rhizomes | |
| Color: | Near 155D |
| Size: | About 10-25 cm |

THE BUD STAGE

| | |
|---|---|
| Form: | Pearshaped |
| Size | |
| Diameter: | About 0.5-1 cm |
| Length: | About 1-2 cm |
| Rate of opening: | Fast |
| Color of pepals when | Near 137C |
| tepals begun to unfurl: | |

THE FLOWER STAGE

| | |
|---|---|
| Blooming habit: | Recurrent, year-round |
| Flower | |
| Size: | Moderate |
| Diameter | About 5-6 cm |
| Depth: | About 4-5 cm |
| Born: | Singly along simple |
| | racemes |
| Inflorescence | |
| Shape: | Umbel when bloom first |
| | opens and matures |
| Peduncle or flower stem | |
| Length: | About 7-15 cm |
| Color: | Near 137C |
| Strength: | Strong |
| Diameter: | About 0.5 cm |
| Discoloration: | No discoloration after |
| | full bloom |
| Florets per stem: | About 3-5 |
| Hairy: | Not hairy |
| Bloom | |
| Type: | Single |
| Form: | Cup |
| Borne: | Singly along simple |
| | racemes |
| Petalage: | About 6 |
| Shape of tepals: | Obovate |
| Arrangement: | Two concentric circles of |
| | 3 tepals |
| Margin: | Some serration |
| Apex: | Pointed |
| Size of bloom fully | |
| expanded | |
| Diameter: | About 5-6 cm |
| Depth; | About 4-5 cm |
| Shape: | Cup |
| Texture: | Soft |
| Aspect: | Flower does not hang on |
| | and dry |
| Lasting quality: | About 21 days on plant; |
| | about 14 days after |
| | cutting |
| Color: | Near 155D; outer tepals |
| | have green tip near 137C; |
| | outer tepals bear no |
| | stripes; inner lateral |
| | tepals near 13B; inner |
| | lateral tepals bear 20-30 |
| | spots near 185A; inner |
| | media tepals have about |
| | 5-15 spots near 185A and |
| | 4-8 spots near 13C. |
| Point of tepal | Near 155D |
| attachment: | |
| Discoloration as | No discoloration |
| bloom ages: | |
| Fragrance: | None |

REPRODUCTIVE ORGANS

| | |
|---|---|
| Stamen | |
| Number: | About 6 |
| Color: | Near 155D |
| Arrangement: | Around the style |
| Anthers | |
| Size: | About 0.5 to 1 cm |
| Color | Near 138A |
| Filaments | |
| Length: | About 5 cm |
| Color: | Near 155D |
| Pollen | |
| Quality: | Good |
| Color: | Near 152C |
| Pistils | 1 |
| Styles: | Length about 4-6 cm |
| Color: | Near 64C |
| Stigmas | |
| Color: | Near 13C |
| Fertile: | Yes |
| Fruit | |
| Shape: | Round |
| Size: | About 1-2 cm |
| Color at maturity: | Near 164A |

I claim:

1. A new and distinct Alstroemeria plant of the variety substantially as shown and described.
